(19) United States
(12) Patent Application Publication
(10) Pub. No.: US 2025/0185901 A1
(43) Pub. Date: Jun. 12, 2025

(54) ANGLED HEAD ASSEMBLY FOR SURGICAL CAMERA

(71) Applicant: Arthrex, Inc., Naples, FL (US)

(72) Inventors: Andrew Melton, Santa Barbara, CA (US); Tzung-Yu Hsu, Taoyuan City (TW); Benjamin Hedges, Santa Barbara, CA (US); Ethan Franklin, Santa Barbara, CA (US); Zachary Dominguez, Carpinteria, CA (US); Jason Metzner, Snoqualmie, WA (US); Connor Engstrom, Ventura, CA (US)

(73) Assignee: Arthrex, Inc., Naples, FL (US)

(21) Appl. No.: 18/976,850

(22) Filed: Dec. 11, 2024

Related U.S. Application Data
(60) Provisional application No. 63/608,350, filed on Dec. 11, 2023.

(51) Int. Cl. A61B 1/06; A61B 1/05

(52) U.S. Cl. CPC A61B 1/0623; A61B 1/051

(57) ABSTRACT

A surgical camera apparatus includes an elongated head assembly having a profile shape extending along a longitudinal axis. The elongated head assembly forms an angled distal tip oriented at a tip angle offset from the longitudinal axis. The angled distal tip may house a camera module and a light source in a stacked configuration.

### CROSS-REFERENCE TO RELATED APPLICATIONS

This application claims priority under 35 U.S.C. § 119(e) and the benefit of U.S. Provisional Application No. 63/608,350 entitled ANGLED HEAD ASSEMBLY FOR SURGICAL CAMERA, filed on Dec. 11, 2024, by Andrew Melton, et al., the entire disclosure of which is incorporated herein by reference.

### BACKGROUND

Minimally invasive procedures generally provide for improved patient outcomes by limiting tissue damage necessary to access a surgical site. To practice such procedures, various devices may be implemented with elongated probes and remote imaging capabilities to access patient cavities for treatment. The disclosure provides improvements for imaging devices that may be used for minimally invasive procedures.

### SUMMARY

In various implementations, the disclosure provides for a surgical camera apparatus that may provide improved access for capturing image data in patient cavities and/or for use in coordination with various surgical tools. The camera apparatus may incorporate a distal camera module that may be housed within an elongated tubular housing or enclosure and accompanied by a light source. The camera module may be disposed at a distal tip of the tubular housing and may be formed within a sealed closure incorporating a proximal image sensor spaced apart from a distal lens over a focal length. In this configuration, the camera module and light source may be housed within the distal end portion of the tubular housing to illuminate a remotely accessed imaging scene and capture corresponding image data in a field of view of the camera module for various applications. While improvements in technology and manufacturing processes may support decreased package sizes for the camera module and the light source, the head assembly of the camera apparatus may be provided in a stacked configuration relative to the geometry of the apparatus to limit the proportions of the head assembly while improving the performance of the camera apparatus.

In various implementations, the head assembly may extend along a longitudinal axis and have a profile shape terminating at an angled distal tip. The profile shape of the head assembly may be elongated having a height greater than the width. When applied in combination, a distal face formed by the angled distal tip at the tip angle, the elongated proportions of the profile shape may be increased as a result of the angled distal tip extending diagonally through the height of profile shape at the tip angle. In this configuration, the resulting distal face may be extended in height along the effective hypotenuse of the angled distal tip. This elongation of the height may increase a surface area of the distal face, thereby accommodating an increased imaging surface for the camera module and emission surface of the light source.

These and other features, objects and advantages of the present disclosure will become apparent upon reading the following description thereof together with reference to the accompanying drawings.

### BRIEF DESCRIPTION OF THE DRAWINGS

FIG. 1A is a projected view of a camera apparatus demonstrating an elongated head assembly having an angled distal tip;

FIG. 1B is a side profile view of an optical head assembly for the camera apparatus demonstrated in FIG. 1A;

FIG. 1C is a projected view of the optical head assembly demonstrated in FIGS. 1A and 1B implemented in a sheath assembly;

FIG. 1D is a front view of the optical head assembly demonstrated in FIGS. 1A and 1B implemented in a sheath assembly;

FIG. 2A is a projected view demonstrating a tubular housing having a profile shape extending along a longitudinal axis of the elongated head assembly and an angled distal tip;

FIG. 2B is a front view of the tubular housing demonstrating an exemplary profile shape;

FIG. 2C is a front view of the tubular housing demonstrating an exemplary profile shape;

FIG. 2D is a front view of the tubular housing demonstrating an exemplary profile shape;

FIG. 3A is a projected view of a camera apparatus demonstrating an elongated head assembly having an angled distal tip;

FIG. 3B is a front view of the optical head assembly demonstrated in FIGS. 1A and 1B implemented in a sheath assembly;

FIG. 3C is a side cross-sectional view of an optical head assembly for the camera apparatus demonstrated in FIG. 1A;

FIG. 4A is a projected view of an exemplary connection configuration of a camera module and light source;

FIG. 4B is a projected view of an exemplary connection configuration of a camera module and light source;

FIG. 4C is a projected view of an exemplary connection configuration of a camera module and light source;

FIG. 4D is a projected view of an exemplary connection configuration of a camera module and light source;

FIG. 4E is a projected view of an exemplary connection configuration of a camera module and light source;

FIG. 5A is a pictorial view of a step of a manufacturing process for forming a camera module and light source in a potted assembly;

FIG. 5B is a pictorial view of a step of a manufacturing process for forming a camera module and light source in a potted assembly;

FIG. 5C is a pictorial view of a step of a manufacturing process for forming a camera module and light source in a potted assembly;

FIG. 5D is a pictorial view of a step of a manufacturing process for forming a camera module and light source in a potted assembly;

FIG. 5E is a pictorial view of a step of a manufacturing process for forming a camera module and light source in a potted assembly;

FIG. 6A is a pictorial view of a step of a manufacturing process for forming a camera module and light source in a potted assembly;

FIG. 6B is a pictorial view of a step of a manufacturing process for forming a camera module and light source in a potted assembly;

FIG. 6C is a pictorial view of a step of a manufacturing process for forming a camera module and light source in a potted assembly;

FIG. 6D is a pictorial view of a step of a manufacturing process for forming a camera module and light source in a potted assembly;

FIG. 7A is a pictorial view of a step of a manufacturing process for forming a camera module and light source in a potted assembly;

FIG. 7B is a pictorial view of a step of a manufacturing process for forming a camera module and light source in a potted assembly;

FIG. 7C is a pictorial view of a step of a manufacturing process for forming a camera module and light source in a potted assembly;

FIG. 7D is a pictorial view of a step of a manufacturing process for forming a camera module and light source in a potted assembly;

FIG. 8A is a pictorial view of a step of a manufacturing process for forming a camera module and light source in a clamshell assembly;

FIG. 8B is a pictorial view of a step of a manufacturing process for forming a camera module and light source in a clamshell assembly;

FIG. 8C is a pictorial view of a step of a manufacturing process for forming a camera module and light source in a clamshell assembly;

FIG. 9A demonstrates an exemplary step of a manufacturing process for positioning a camera module and light source with a locating assembly;

FIG. 9B demonstrates an assembly process of a locating assembly inserted into a tubular housing of an elongated head assembly;

FIG. 9C demonstrates an alternate configuration of the locating assembly demonstrated in FIG. 9A;

FIG. 9D demonstrates an alternate configuration of the locating assembly demonstrated in FIG. 9A;

FIG. 9E demonstrates a profile view of the locating assembly of FIG. 8D;

FIG. 10A is an exemplary step of the manufacturing process demonstrating the positioning of a camera module and light source in a preformed distal housing;

FIG. 10B is an exemplary step of the manufacturing process demonstrating the positioning of a camera module and light source in a preformed distal housing;

FIG. 10C is an exemplary step of the manufacturing process demonstrating the positioning of a camera module and light source in a preformed distal housing;

FIG. 10D is an exemplary step of the manufacturing process demonstrating the positioning of a camera module and light source in a preformed distal housing; and

FIG. 11 is a block diagram demonstrating an imaging system incorporating at least one surgical camera apparatus in accordance with the disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

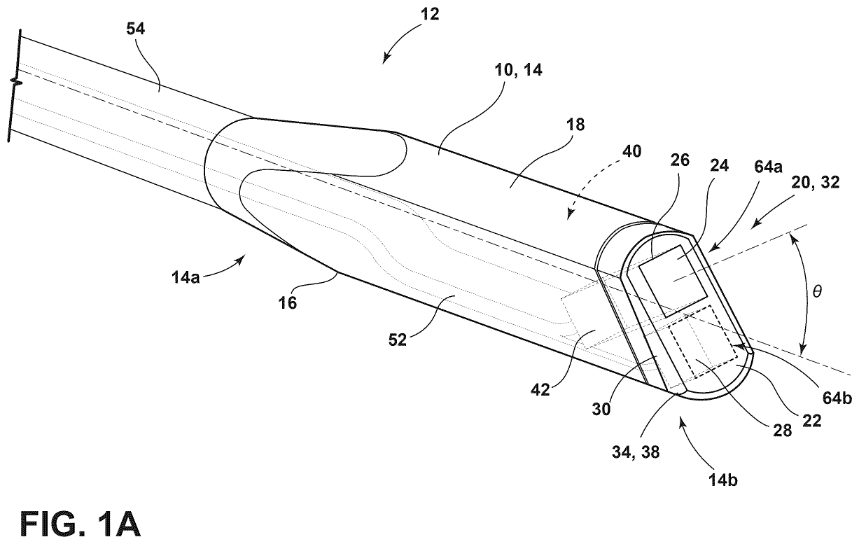

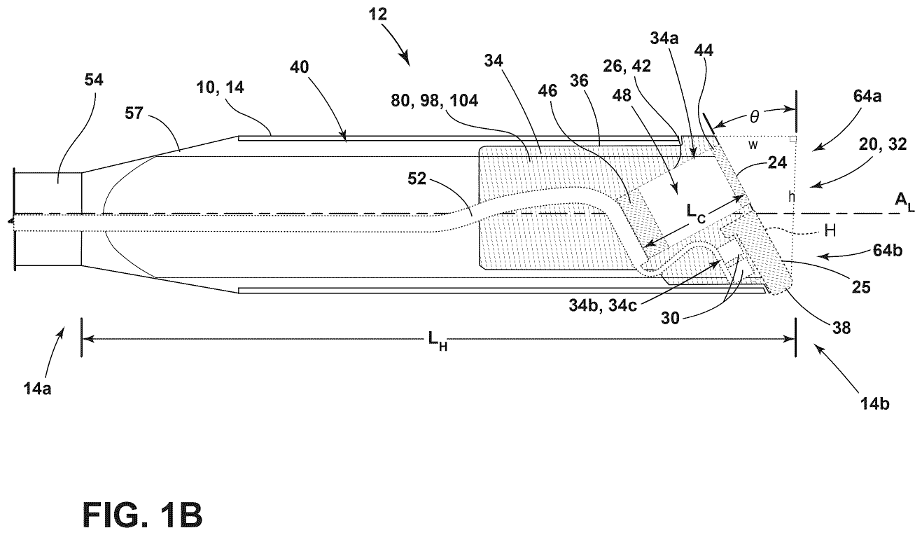

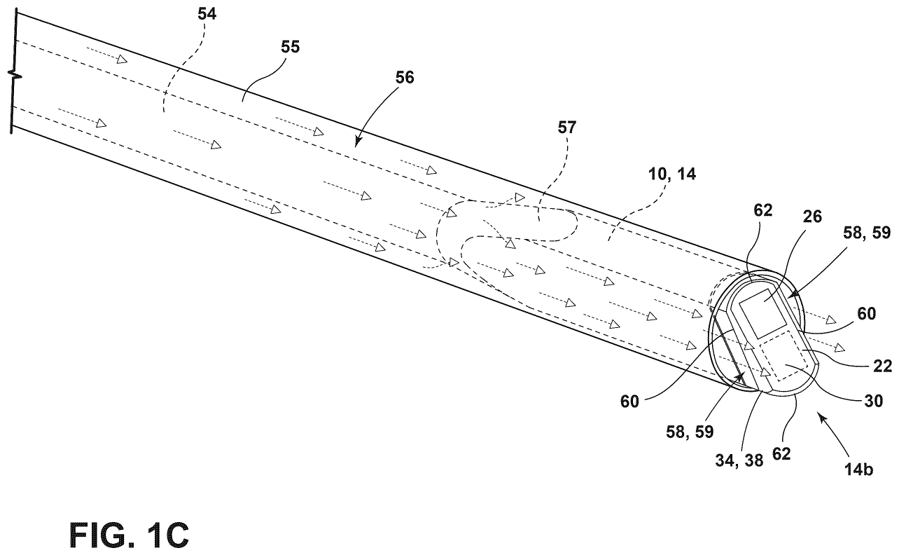

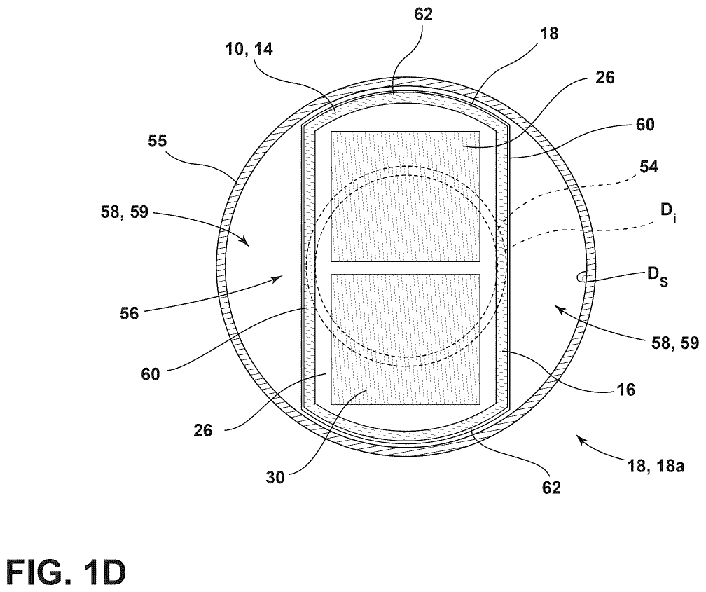

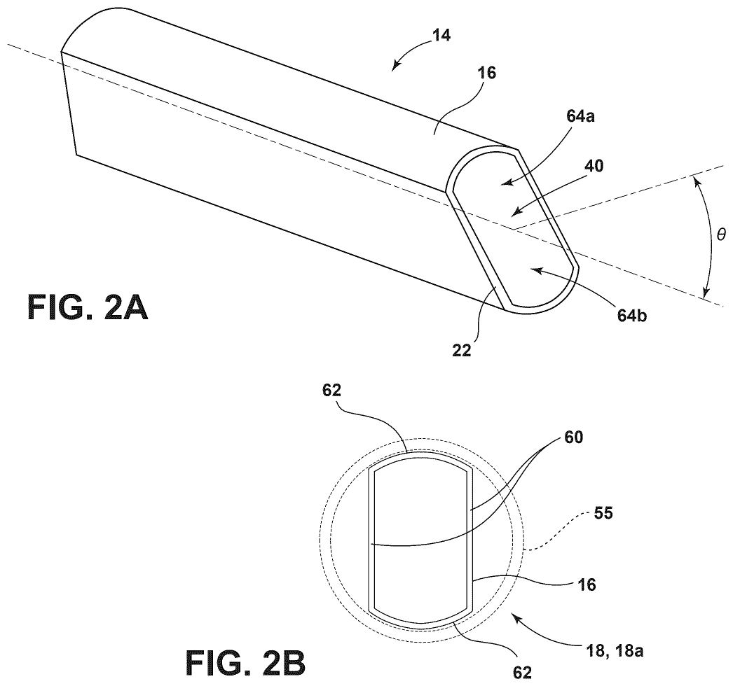

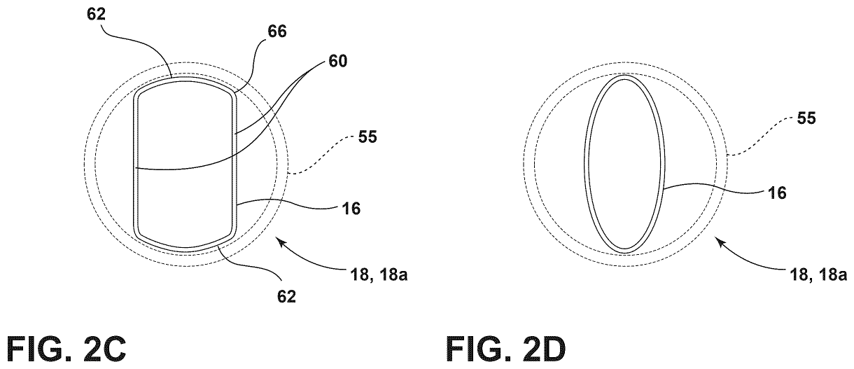

Referring generally to FIGS. 1 and 2, the disclosure may provide an optical head assembly 10 for a surgical camera apparatus 12. As shown in FIGS. 1A and 1B, the head assembly 10 may comprise an elongated body 14 extending over a length L_{H} along longitudinal axis A_{L} from a proximal end portion 14a to a distal end portion 14b. The elongated body 14 may be formed by a tubular housing 16 that may form a profile shape 18 that extends along the longitudinal axis A_{L}. The distal end portion 14b may form a distal tip 20 oriented or pitched at a tip angle θ that may be offset from the longitudinal axis A_{L} forming a pitched or angled distal face 22. In this configuration, the distal face 22 may accommodate an optical surface 24 of a camera module 26 and an emission surface 28 of a light source 30. The distal face 22 may accommodate the emission of light from the light source 30 and reception of light representing a scene in a field of view of the camera module 26 captured over the optical surface 24.

The positioning of the camera module 26 and the light source 30 along the distal face 22 may be accommodated by an elongation of the profile shape 18 resulting from the intersection between the profile shape 18 and the tip angle θ forming the distal tip 20. The elongation of the distal face 22 may be the result of the tip angle of the angled tip extending through a height of the profile shape 18. In this configuration, the resulting distal face 22 may be extended in height along the effective hypotenuse 34 of the angled distal tip 20. Additionally, in some implementations, the profile shape 18 of the head assembly may be greater in height than in width. The elongated height of the profile shape 18 may be aligned with the direction or pitch of the tip angle θ relative to the longitudinal axis A_{L}. In this configuration, the elongated profile shape 18 may be further elongated by the tip angle θ extending through the profile shape 18. The elongation of the profile shape 18 along the tip angle θ may allow distal face 22 to accommodate the camera module 26 and the light source 30 in a stacked configuration 32, allowing the optical head assembly 10 to be implemented in an angled configuration at the tip angle θ while also limiting the resulting package size of the camera apparatus 12 for improved access and deployment in various applications.

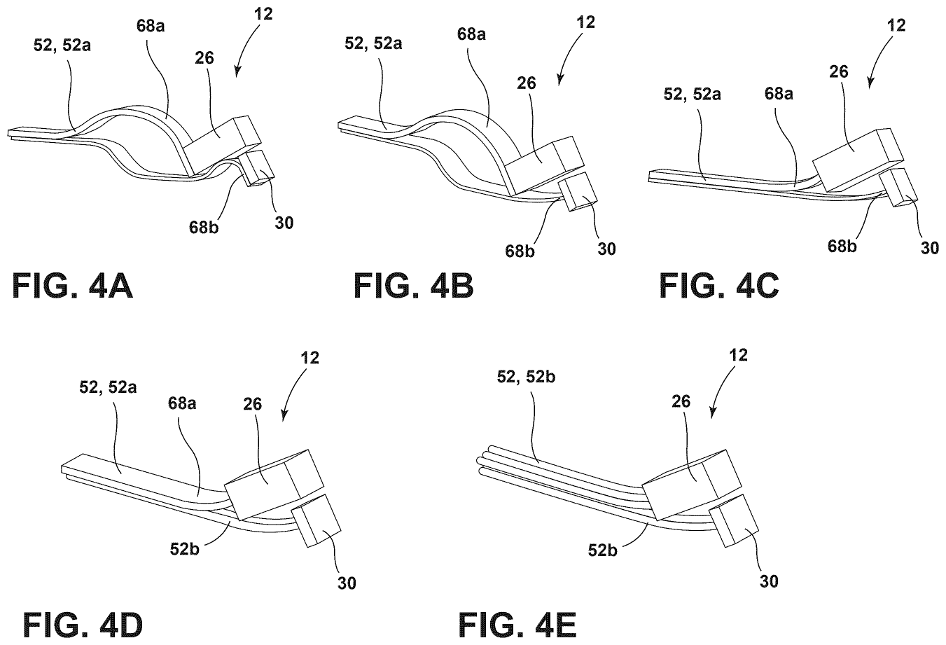

As best demonstrated in FIG. 1B, the camera module 26 and light source 30 may be retained in a distal insert housing 34 that may correspond to a potted assembly 80 as later discussed in reference to FIGS. 4-8. In such implementations, the insert housing 34 may be inserted into a distal opening of the tubular housing 16 and engage a distal end portion of the tubular housing 16. The insertion of the insert housing 34 into the tubular housing may provide for the formation of a seal surface 36 that may extend distally to a peripheral ridge 38 that may engage the distal end of the tubular housing 16 in an assembled configuration. In this configuration, the seal surface 36 may provide for improved engagement and sealing of the insert housing 34 within the distal end of the tubular housing 16. In various implementations the seal surface 36 may be adhered, welded, fused, or otherwise sealably connect the insert housing 34 within the tubular housing 16, thereby securing the camera module 26 and light source 30 within the head assembly 10.

Still referring to FIGS. 1 and 2, the camera module 26 and light source 30 may extend along the tip angle θ of the distal face 22 and into an interior volume or interior cavity 40 of the tubular housing 16. As best demonstrated in FIG. 1B, the light source 30 may be positioned distally of the camera module 26 along the tip angle θ formed by the distal face 22. The camera module 26 may be positioned proximal of the light source 30 away from the narrow, distal tip 20. Accordingly, a length L_{C} or depth of a camera closure 42 of the camera module 26 may extend from the distal face 22 along the tip angle θ into the interior cavity 40. In this configuration, the length L_{C} of the camera closure 42 may extend diagonally into the interior cavity 40 proximal of the light source 30, thereby accommodating the comparatively increased length or depth of the camera module 26 relative to the light source 30 within the interior cavity 40.

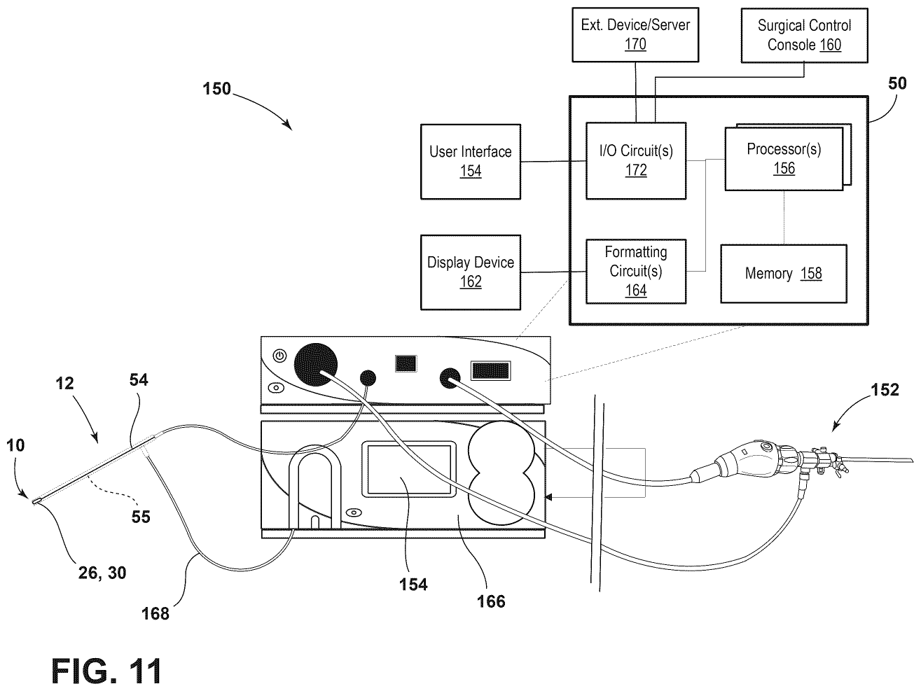

As previously discussed, the optic surface 24 of the camera module 26 may be aligned with the distal face 22 of the optical head assembly 10 along the tip angle θ. Within the camera closure 42, the camera module 26 may comprise an imaging lens 44 positioned proximate to the optic surface 24 and at least one image sensor 46 separated from the imaging lens 44 over a focal length 48. The camera module 26 may correspond to a chip-on-tip or chip-type camera design configured to capture and communicate image data presenting a scene within a field of view of the imaging lens 44 to an imaging controller 50 (see FIG. 11) via at least one conductive connector 52. The conductive connector 52 may extend through or interconnect with a communication cable or communication interface 54 that may have decreased proportions relative to the profile shape 18 of the head assembly 10. Though not shown in FIGS. 1A and 1B, the camera module 26 may further incorporate one or more filters, including a color filter array and bandpass filters that may be required to accurately capture full color images.

As shown in FIG. 1B, the at least one conductive connector 52 may correspond to a flex-circuit or flexible printed circuit (FPC) providing control and communications for the camera module 26 and the light source 30 along a common communication interface. In the example shown, the camera module 26 is connected to the conductive connector 52 proximal of the light source(s) 30 providing for an economical interface that may improve manufacturability of the head assembly 10. The light source 30 may correspond to at least one light emitting diode (LED) enclosed within the tubular housing 16 and operably coupled in a pocket formed in the insert housing 34. In this configuration, the light emissions output from the light source(s) 30 may illuminate a scene in the field of view of the camera module 26. The light output from the light source 30 may correspond to one or more wavelengths of light that may include a visible light spectrum and/or other spectrums of light including a near infrared or infrared light spectrum. In an exemplary embodiment, the light source 30 may be configured to output a substantially white light or color tunable lighting emission to illuminate the scene captured within the field of view of the camera module 26.

In various implementations, the insert housing 34 may include an at least partially transparent material extending over and forming the emission surface 28 on the distal tip 20. For example, all or part of the insert housing 34 may be formed by an optically transmissive material that may allow the light emissions from the light source(s) 30 to be transmitted through the material of the insert housing 34. In this configuration, the distal housing may form a continuous surface extending over the distal face 22 and sealing the light source(s) 30 within the optical head assembly 10. As shown, an opening 34a may be formed through the distal face 22 of the insert housing 34 through which the camera module 26 may extend adjacent to the light source 30. Additionally, a recessed pocket 34b may be formed in an interior surface 34c of the insert housing 34 in which the light source(s) 30 may be operably coupled to the light-transmissive material forming the distal tip 20 of the insert housing 34. In this configuration, the light transmissive material forming the distal tip 20 may transmit light emitting from the light source(s) 30 illuminating the field of view of the camera module 26.

As shown in FIGS. 1C and 1D, in some implementations, the optical head assembly 10 may be implemented in a sheath assembly 55 (e.g., an outflow sheath) configured to communicate fluid along a length of the camera apparatus 12. As shown in FIG. 1C, the head assembly 10 is disposed within a flow passage 56 of the sheath 55. In this configuration, a fluid flow (e.g., a saline solution) represented by arrows in FIG. 1C may be delivered to the distal tip 20 via a perimeter gap 56a formed between the profile shape 18 of the elongated body 14 and a sheath diameter Dₛ of the cylindrical interior of the flow passage 56 formed by the sheath 55. The delivery of the fluid to the distal tip 20 may be facilitated by providing the communication interface 54 having an interface diameter Dᵢ that is smaller in proportion (e.g., directionally or vertically) than the sheath diameter Dₛ. The proximal end portion 14a of the elongated body 14 may form a tapered transition portion 57. The transition portion 57 may correspond to a smooth drafted section extending between the interface diameter Dᵢ at the proximal end 14a of the body 14 to the profile shape 18 at an intermediate portion 14c of the body 14. In this way, the fluid flow may be delivered effectively through the flow passage 56 to the head assembly 10.

Beyond the length of the communication interface 54 of the apparatus 12, the fluid flow may be diverted from the annular path formed between the interface diameter Dᵢ and the sheath diameter Dₛ along the transition portion 57 to opposing flow passages 58 formed between the profile shape 18 and the sheath diameter Dₛ of the interior flow passage 56. The opposing flow passages 58 may extend to a fluid outlet 59 of the sheath 55 along opposing elongated sides 60 of the head assembly 10, which may be aligned with the height of the distal face 22 along the tip angle θ angle of the distal face 22. In this configuration, the fluid flow along the length of the communication interface 54 may be supplied along a first cross-sectional flow area A_{f1} that may be greater than a second cross-sectional flow area A_{f2} extending between the opposing elongated sides 60 of the head assembly 10 and the sheath diameter Dₛ of the flow passage 56. This configuration may limit the pressure drop of the fluid flow delivered along an extended length of the communication interface 54. In this way, the pressure of the fluid may be supplied through the flow passage 56 to the head assembly 10 and effectively delivered to the fluid outlet 59 while minimizing the overall clearance diameter D_{c} defined by proportion of the sheath 55.

In various implementations, surgical camera apparatus 12 may be configured to capture imager data with the camera module 26 at the tip angle θ offset or pitched from the longitudinal axis A_{L}. As shown, the tip angle θ may be approximately 30° and may vary from approximately 25° to 35°. In some implementations, the tip angle θ may be accommodate addition viewing angles that may range from approximately 10° to 45° and in some cases may provide for viewing angles exceeding 45° including angles of 60°, 70°, or more. The image sensor 46 may correspond to a complimentary metal-oxide-semiconductor sensor that may have a resolution approximately 700×700 pixels. In general, the imager sensor 46 may be implemented with a variety of resolutions including 200×200, 400×400, 700×700, 1000×1000 or higher resolutions. It shall be understood that the resolution of the image sensor 46 may be limited based on the package size of the camera closure 42 and the camera module 26. However, technological advances continue to drive package size down, which may allow the camera apparatus 12 to achieve even higher resolutions while limiting the package size to proportions similar to or even smaller than those discuss in reference to FIG. 2 in the following description. Though illustrated in various exemplary configurations, the profile shape 18 of the camera apparatus 12 may be implemented in various forms including an elliptical profile, rectangular profile, or square profile, or various irregular shapes and complex geometries that may provide similar operating configurations to those described herein.

Referring now to FIGS. 2A and 2B, the tubular housing 16 is shown demonstrating an exemplary first profile shape 18a that may exemplify the elongated profile further extended in height along the tip angle θ formed by the distal face 22. In the example shown, the profile shape 18a may include laterally opposing straight sides 60 and vertically opposed curved or convex sides 62. In this configuration, the distal face 22 may proportionally be split along the longitudinal axis A_{L} into an upper region 64a and a lower region 64b for spatial reference. In the orientation shown in FIG. 2A, the lower region 64b may correspond to a narrow tip portion of the distal tip 20. The lower region 64b may correspond to a narrowed or diminished portion of the interior volume of the interior cavity 40 and may accordingly be limited in depth in the normal direction extending from the distal face 22 into the interior cavity 40 relative to the upper region 64a. As provided in various exemplary configurations, the light source 30 may be housed within the volume formed by the interior cavity 40 of the tubular housing 16 substantially over the lower region 64b corresponding to the narrow portion of the distal tip 20. The stacked configuration 32 may further provide for the camera module 26 to be positioned substantially along the upper region 64a of the distal face 22 opposite the tapered end portion of the distal tip 20. The position of the camera module 26 substantially in the upper region 64a may ensure that the camera length L_{C} of the camera closure 42 may extend diagonally into the interior cavity 40 of the tubular housing 16 along the tip angle θ. As demonstrated in FIG. 2B, the exemplary dimensions of the height, width, thickness, and curvatures of the profile shape 18 are demonstrated in millimeters for reference. However, it shall be understood that the proportions of the tubular housing 16 and corresponding proportions of the optical head assembly 10 may vary among applications.

Referring now to FIGS. 2C and 2D, additional examples of the profile shape 18 of the tubular housing 16 are shown. In FIG. 2C, a second profile shape 18b is shown may be similar to the first profile shape 18a from FIG. 2B but may further include curved edges 66 extending between the laterally opposing straight sides 60 and the vertically opposed curved, convex sides 62. In this configuration, the second profile shape 18b may form a smooth exterior perimeter surface. As demonstrated in FIG. 2D, a third profile shape 18c may correspond to an ovular, elliptical, or oblong circular shape. In such implementations, the geometry defining the third profile shape 18c may be defined by a vertical radius and a horizontal or lateral radius that may generally correspond to the height and width of the profile shape 18 as previously discussed. In various implementations, the profile shape 18 may be elongated in the direction corresponding to the tip angle θ forming the distal tip 20 (e.g., the height as shown). As a result, the geometry of the profile shape 18 may further be elongated based on the directional cross-section defined by the tip angle θ, which may correspond to a hypotenuse H formed by the angled or cut-away portion of distal face 22.

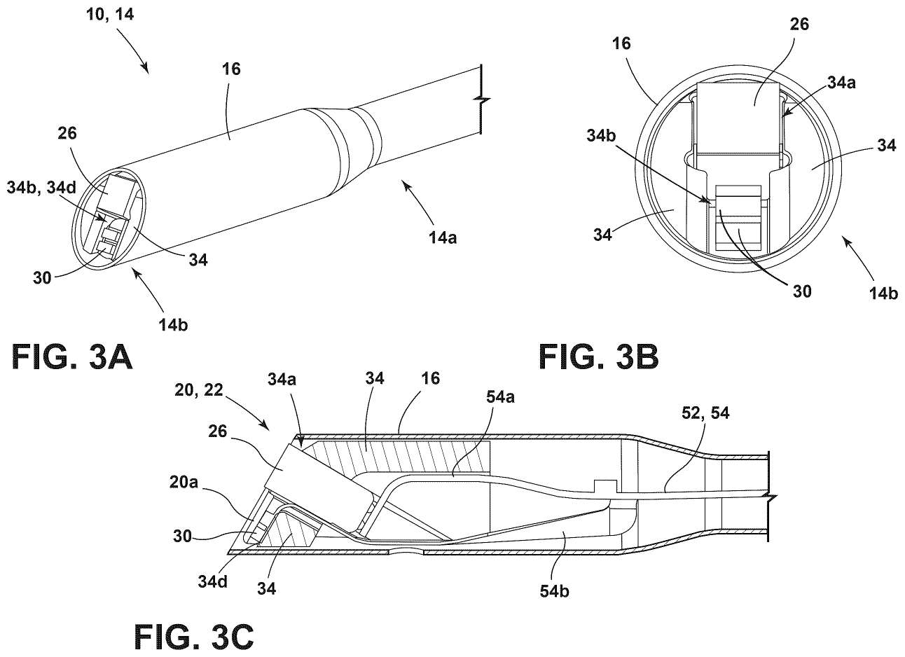

Referring now to FIGS. 3A, 3B, and 3C, another exemplary implementation of the optical head assembly 10 is shown. In the example shown, the tubular housing 16 is substantially circular or elliptical and includes a similar distal tip 20 formed by a light transmissive material of the insert housing 34 forming the distal face 22. In the example shown, the portion of the insert housing 34 forming the distal tip is not readily apparent in FIGS. 3A and 3B. These representations emphasize the light transmissive or transparent nature of the distal tip 20 through which the light emission from the light source(s) is output. The cross-sectional view of the optical head assembly 10 demonstrates the distal tip 20 formed at a lens extending over a portion of the distal face 22 through which light emitted from the light source(s) is transmitted to illuminate the field of view of the camera module 26. Accordingly, the distal tip 20 may be formed as a portion of the insert housing 34 and/or as a light transmissive lens or cover portion 20a as shown in FIG. 3C.

As additionally shown in FIGS. 3A-3C, the insert housing 34 is positioned within the tubular housing 16, such that the distal face 22 is recessed within the distal opening of the tubular housing 16. In this configuration, the insert housing 34 may be nested within the distal opening of the tubular housing 16 as best illustrated in FIG. 3C. In contrast with the example shown in FIG. 1, the recessed pocket 34b may be formed in an exterior surface 34d of the insert housing 34. In this configuration, the cover portion 20a may form a light transmissive body enclosing the distal face 22 over the light source(s) 30. As shown, the cover portion 20a may form a bridged surface extending across the recessed pocket 34b, which may form a seal enclosing the light source(s) in the distal tip 20 of the optical head assembly 10. In various implementations, the insert housing 34 may include features configured to received or on which the camera module 26 and the light source(s) 30 may be operably coupled to the tubular housing 30 limiting variations in the manufacture of the optical head assembly 10.

As further demonstrated in FIG. 3C, the conductive connector 52 may be split within the tubular housing 16 near the proximal end portion 14a of the elongated body 14 and include a separate camera connection interface 54a and light connection interface 54b. The conductive connector 52 forming the communication interfaces 54a, 54b may similarly be implemented as a flex circuit or FPC that may limit complexity in assembly of the head assembly. The separate interfaces 54a, 54b may be combined to form a common section 54c of the communication interface extending from the optical head assembly 10 to a control console or camera controller. The variations in the communication interface 54 disclosure may support a wide variety of configurations of the head assembly 10 including various constructions, some of which are shown in FIGS. 4-10. As discussed herein the flex circuit or conductors forming the communication interface 54 may be formed or one or more flexible substrates (e.g., polyamides) comprising conductive layer (e.g., copper films) forming the individual conductive connections. Though specific materials and configurations are provided, these examples are not limiting to the scope of the disclosure.

Referring now to FIGS. 4A-4E, exemplary configurations of the at least one conductive connector 52 are shown in connection with the camera module 26 and the light source 30. In general, the conductive connector 52 may correspond to one or more flexible printed circuits that may be formed by printing of one or more conductors and coating the conductors in insulating material. The conductive connector(s) 52 may also be formed or molded to specific shapes to accommodate the geometry required to conductively connect the conductive connectors 52 to the camera module 26 and/or the light source 30. Among the examples of the configurations of the conductive connectors 52, flexible printed circuits 52a are implemented in both an edge-mount configuration 68a and a surface-mount configuration 68b. In some applications, one or more of the conductive connectors 52 may be alternately provided by conductive wires 52b that may be interconnected as custom wiring harnesses or implemented as individual wires, as shown. In this way, the optical head assembly 10 may be implemented in a variety of configurations to suit various applications.

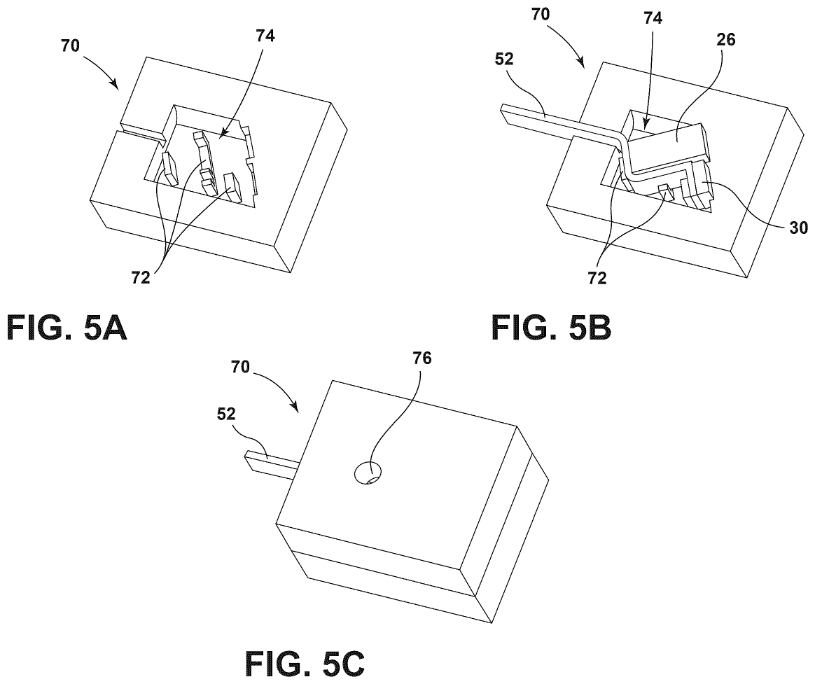

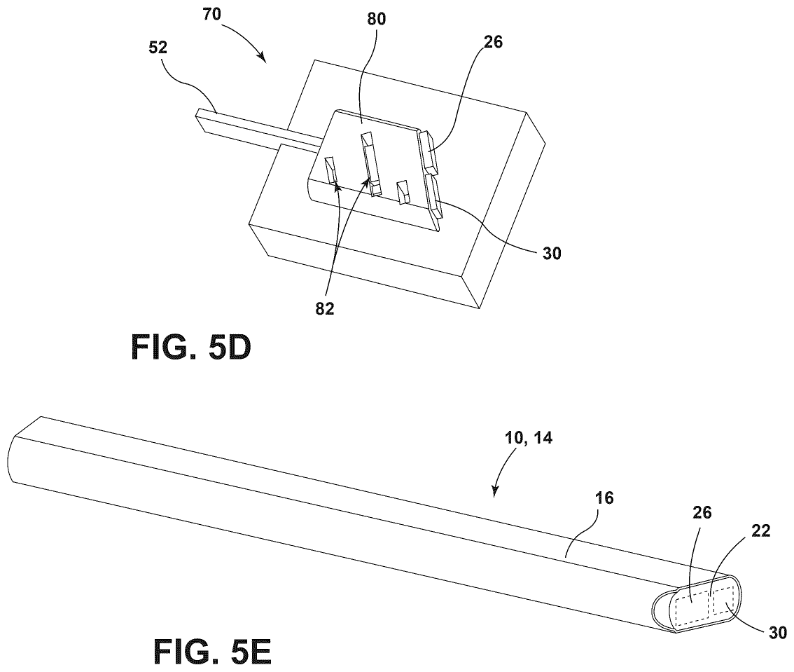

In the example shown in FIG. 5A, the camera module 26 and the light source 30 may be connected to flexible printed circuits 52a in a surface-mount configuration 68b. In FIG. 5B, the camera module 26 is shown connected via a surface-mount configuration 68b and the light source 30 is connected via the edge-mount configuration 68a. Alternatively, the edge-mount and surface-mount configurations 68a, 68b may be connected to the camera module 26 and the light source 30, respectively. In FIG. 4C, both the camera module 26 and the light source 30 are connected via the edge-mount configuration 68a. Finally, as demonstrated in FIGS. 4D and 4E, the flexible printed circuit 52a may be alternatively implemented by the conductive wires 52b in connection with either the camera module 26 and/or the light source 30. Accordingly, the disclosure provides for the conductive connectors 52 to be implemented in a variety of configurations to suit the application of camera apparatus.

As previously discussed in FIGS. 1-4, the camera apparatus may include a variety of features to improve deployment in surgical applications. As described generally in FIGS. 5-10, the camera apparatus 12 may be manufactured using a variety of techniques. The examples in FIGS. 5-10 demonstrate various examples of manufacturing procedures, fixtures, and associated assemblies that may be implemented to manufacture the optical head assembly 10 in various configurations. For example, as demonstrated in FIGS. 5A-5E, a manufacturing process for the head assembly 10 is described in reference to a potting fixture 70 that may comprise a plurality of locating features 72. As shown in FIG. 5B, the camera module 26, light source 30, and the at least one conductive connector 52 may be positioned by the locating features 72 within a molding cavity 74 that may be configured to orient the camera module 26 and light source 30 along the distal face 22 at the tip angle θ. Once positioned within the molding cavity 74, the camera module 26, light source 30, and the conductive connector 52 may be enclosed within the potting fixture 70 and a potting compound may be injected into an inlet passage 76 of the potting fixture 70 to encapsulate the camera module 26, light source 30, and conductive connectors 52 in an intermediate potted assembly 80. The potted assembly 80 is demonstrated as being partially removed from the molding cavity 74 of potting fixture 70 in FIG. 5D.

The intermediate potted assembly 80 may include one or more apertures or access ports 82 that may be utilized to access portions of the camera module 26, light source 30, and/or the at least one conductive connector 52 following the encapsulation process. The potted assembly 80 may be inserted into the interior cavity 40, such that the optic surface 24 of the camera module 26 and the emission surface 28 of the light source 30 are exposed along the distal face 22 and positioned at the distal tip 20 of the tubular housing 16. Once positioned, the potted assembly 80 may further be potted and/or sealed within the tubular housing 16 to prevent contaminants from reaching the camera module 26, light source 30, and/or the connection(s) to the at least one conductive connector 52.

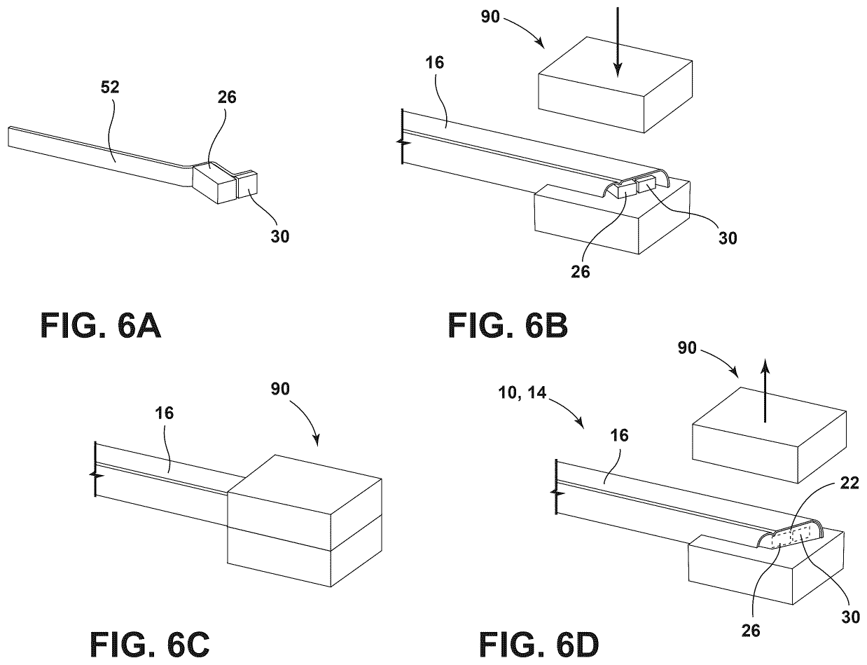

As demonstrated in FIGS. 6A-6D, another exemplary assembly method is shown that may similarly encapsulate the camera module 26, light source 30, and conductive connector 52 within the tubular housing 16. The process demonstrated in FIGS. 6A-6D may differ from that described in reference to FIG. 5 primarily in that the camera module 26 and light source 30 are not initially encapsulated within the intermediate potted assembly 80. For example, as shown in FIG. 6B, the camera module 26, light source 30, and the at least one conductive connector 52 may be inserted and positioned within the interior cavity 40 of the tubular housing 16 prior to encapsulation. With the camera module 26 and light source 30 disposed within the tubular housing 16, the optical head assembly 10 may be positioned within a potting fixture 90. In this configuration, a potting compound may be injected into the potting fixture 90 to concurrently position the camera module 26 and light source 30 within the interior cavity 40 and seal the tubular housing 16 into the encapsulated optical head assembly 10 demonstrated in FIG. 6D. In this way, the optical head assembly 10 may be formed without first implementing the intermediate potted assembly 80 as discussed in reference to FIG. 5.

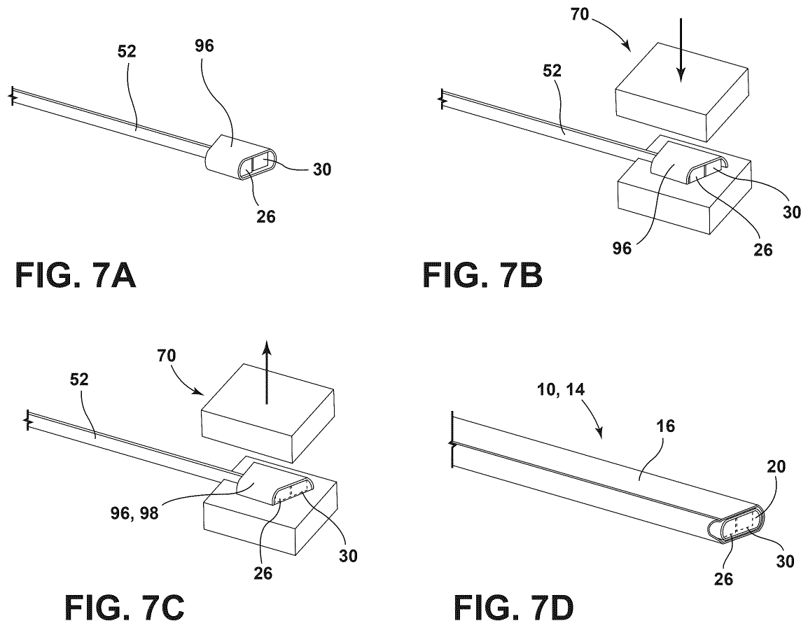

As shown in FIGS. 7A-7D, the camera module 26 and light source 30 may be located within an interior positioning tube 96 that may include locating features 72 similar to those discussed in reference to the potting fixture 70. In this configuration, the interior positioning tube 96 may be utilized to house and locate the camera module 26, light source 30, and the at least one conductive connector 52 within an intermediate potted assembly 98. Similar to the intermediate potted assembly 80, the intermediate potted assembly 98 may be inserted into the interior cavity 40 of the tubular housing 16 and sealed or encapsulated to form the optical head assembly 10 as shown in FIG. 7D.

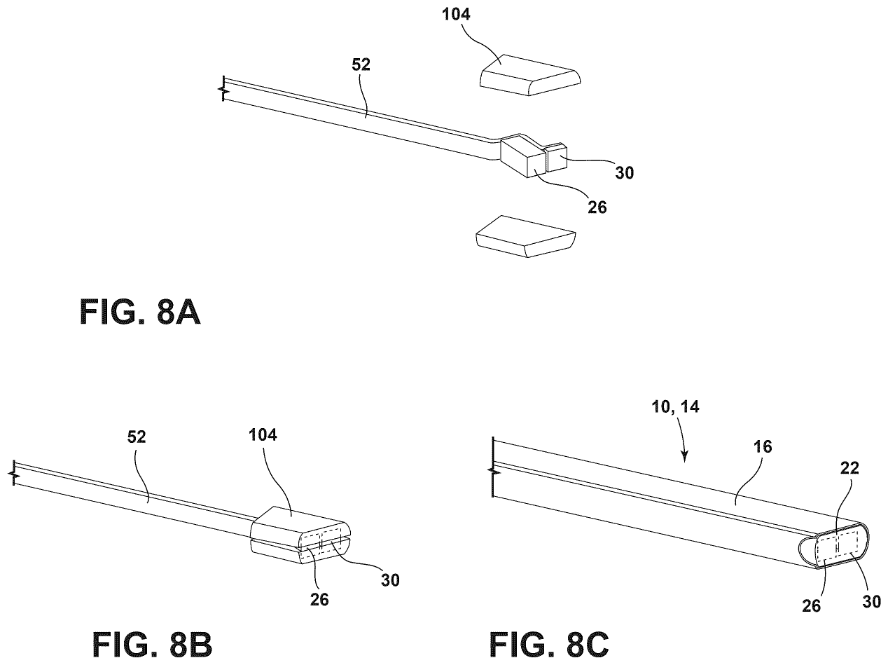

Referring now to FIGS. 8A-8C, the camera module 26, light source 30, and the at least one conductive connector 52 may be initially mounted within a preformed housing insert 104. Similar to the interior positioning tube 96, the preformed housing insert 104 may include one or more locating features 72 that may position the camera module 26 and light source 30 aligned with the distal face 22 at the tip angle θ. As best shown in FIGS. 8A and 8B, rather than forming the intermediate potted assembly 80, 98, the camera module 26 and light source 30 may be secured to the housing insert 104 on opposing sides of a preformed clamshell assembly. The opposing sides of the clamshell assembly may be glued or fused together and positioned within the interior cavity 40 of the tubular housing 16. Accordingly, the camera module 26, light source 30, and the at least one conductive connector 52 may be positioned in a variety of ways prior to insertion into the interior cavity 40 formed by the tubular housing 16.

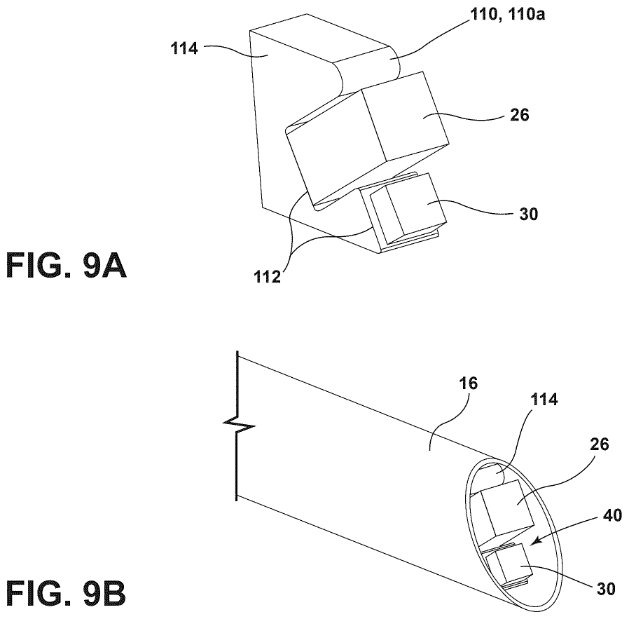

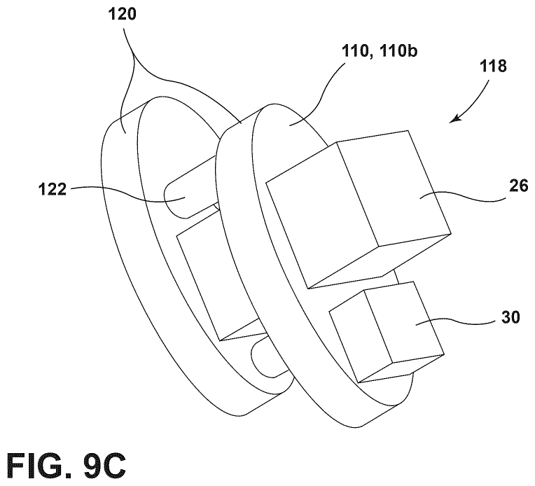

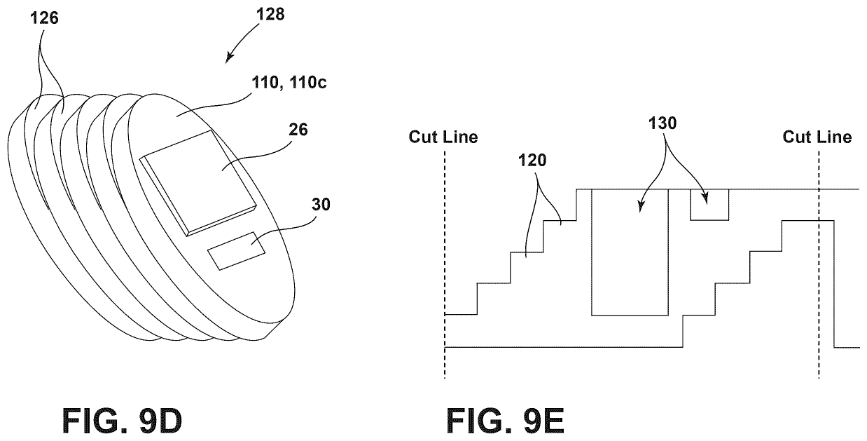

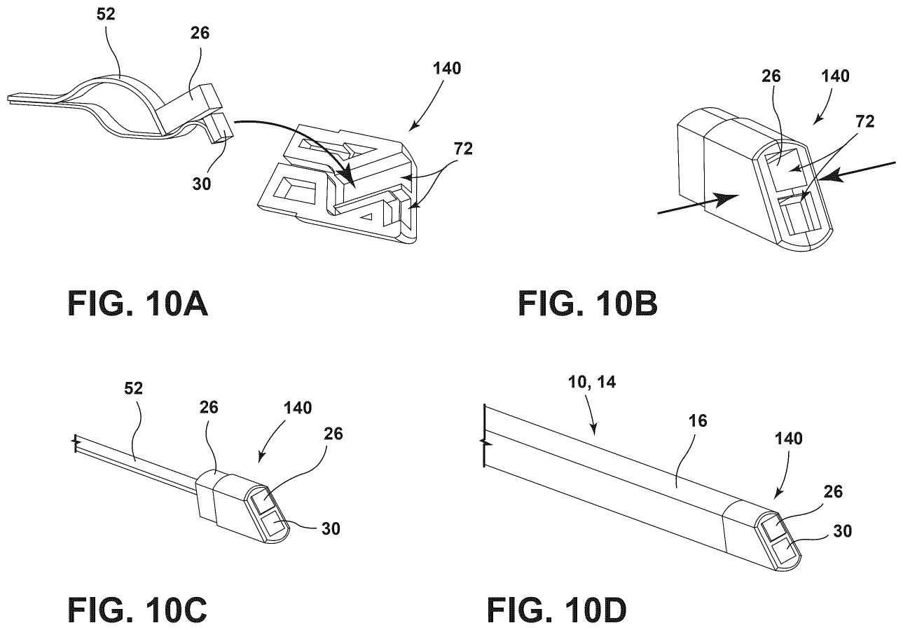

Referring now to FIGS. 9 and 10, additional methods for locating the camera module 26 and light source 30 may include implementing one or more locating assemblies 110. In various implementations, the locating assemblies 110 may include one or more locating features 112 that may be molded, machined, or otherwise formed to receive or locate the camera module 26 and light source 30 for alignment with the distal face 22. Referring first to FIGS. 9A and 9B, a first locating assembly 110a is shown in the form of a circuit substrate 114 (e.g., a three-dimensional printed circuit board and fixture). Similarly, the circuit substrate may be formed as a molded polymeric body comprising one or more traces formed or laminated over the exterior surface of the body. As depicted, the locating features 112 may be formed by a printing process including layers forming the body of the substrate sequentially printed in combination with conductive connections or traces extending through the body aligned with the locating features 112. In this configuration, the locating features 112 may receive the camera module 26, light source 30, and the circuit substrate may incorporate portions of the at least one conductive connector 52. The assembly as shown in FIG. 9A may be positioned within the interior cavity 40. As demonstrated in FIG. 9B, once the camera module 26 and the light source 30 are secured to the molded insert 114, the combined assembly may be positioned within the interior cavity 40 and encapsulated within the tubular housing 16 to form the optical head assembly 10.

FIG. 9C demonstrates another example of the locating assembly 110, referred to as a second locating assembly 110b. As shown, the second locating assembly 110b may be implemented as a plurality of printed circuit boards or circuit substrates 118 that may be positioned in a spaced-apart configuration 120 via one or more connecting spacers 122, which may also provide for conductive connections or vias therebetween. Similar to the circuit substrate 114, the substrates 118 may incorporate a plurality of locating features 112 that may include one or more pockets, apertures, and/or alignment surfaces configured to receive the camera module 26, the light source 30, and/or provide alignment and passage for the at least one conductive connector 52. Accordingly, the locating assembly 110 may be implemented in a variety of ways to position the camera module 26 and the light source 30 in the optical head assembly 10.

As shown in FIGS. 9D and 9E, another implementation of a locating assembly 110 may similarly be implemented with stacked substrates 126 that may be formed of ceramic material and fused together in the stacked configuration 128 shown via a sintering process. The assembly shown in FIG. 9D may be referred to as a third locating assembly 110c for clarity. As demonstrated in FIG. 9E, a side profile of the stacked substrates 126 is shown in the form of a plurality of offset disks that may be staggered in position to form the locating assembly 110 oriented at the tip end 0. In such implementations, the locating features 112 may correspond to one or more locating pockets 130 or apertures that may be sequentially formed in each of the stacked substrates 126. Once connected, the individual cut-outs formed in the substrates 126 may align to receive and position the camera module 26, the light source 30, and/or provide passage for the at least one conductive connector 52.

Referring now to FIG. 10A, in some implementations, the distal tip 20 of the optical head assembly 10 may be formed outside of and extend from the tubular housing 16 in an exposed, preformed housing 140. Similar to the preformed housing insert 104, the exposed preformed housing 140 may incorporate a variety of locating features 72 that may house and position the camera module 26, the light source 30, and/or the at least one conductive connector 52. As shown in FIG. 10B, opposing sides of the exposed preformed housing 140 may enclose the camera module 26 and the light source 30 and may further be sealed or encapsulated within the exposed preformed housing 140. Once encapsulated, a mating or proximal insert portion 142 of the housing 140 may be inserted into the distal end portion 14b of the tubular housing 16. As demonstrated in FIG. 10D, the tubular housing 16 as previously discussed may differ primarily in that the distal tip 20 shown in FIG. 10D is not angled at the tip angle θ. Instead, the tip angle θ of assemblies that utilize the exposed, preformed housing 140 may incorporate the tip angle θ and corresponding elongated profile shape 18 as a portion of the preformed housing 140. In this way, the preformed housing 140 may provide similar features to implementations wherein the tubular housing 16 terminates distally at the tip angle θ. Accordingly, as provided by the various examples described herein, the optical head assembly 10 may be implemented and manufactured in a variety of ways to suit various applications.

Referring now to FIG. 11, a block diagram of an imaging system 150 is shown incorporating the camera apparatus 12. In various implementations, the system 150 may correspond to a video control console in communication with one or more camera devices, which may include a surgical scope 152 and the camera apparatus 12. The system 150 may further be in communication with various surgical tools via the controller 50. The surgical scope 152 may correspond to various devices including an endoscope, laparoscope, arthroscope, etc. As previously discussed, the camera module 26 may correspond to a chip-on-wire imaging device comprising the image sensor 46 and control circuitry incorporated in the camera closure 42 at the distal end portion 14b of the head assembly 10. As demonstrated, the surgical scope 152 and the camera apparatus 12 may be in communication with the controller 50 via the communication interface 54. Though shown connected via a conductive connection, the communication interface 54 may correspond to a wireless communication interface operating via one or more wireless communication protocols (e.g., Wi-Fi, 802.11 b/g/n, etc.).

In various implementations, the light source 30 of the camera apparatus 12 may incorporate one or more of the emitters, which may correspond to various light emitters configured to generate light in the visible range, the near infrared range, or various wavelengths. In various implementations, the emitters may include light emitting diodes (LEDs), laser diodes, or other lighting technologies. The image sensor(s) 46 may correspond to various sensors and configurations comprising, for example, complementary metal-oxide semiconductor (CMOS) sensors, or similar sensor technologies. Though described in reference to specific exemplary technologies, it shall be understood that various aspects of the camera apparatus 12 may be implemented in various applications without being so limited.

In various implementations, the camera module 26 may comprise the control circuitry configured to control the operation of image sensor(s) 46 and/or the emitter(s) of the light source 30. The control circuitry may communicate the resulting image data to the controller 50 for processing prior to recording or display. In various implementations, the camera apparatus 12 may be in communication with a user interface 154, which may include one or more input devices, indicators, displays, etc. The user interface 154 may provide for the control of the camera apparatus 12 including the activation of one or more control routines. The user interface 154 may provide for the selection, adjustment, or toggling of one or more of the image feeds associated with the operation of the camera apparatus 12 and/or the scope 152. The controllers and control circuitry may be implemented by various forms of controllers, microcontrollers, application-specific integrated controllers (ASICs), and/or various control circuits or combinations.

The controller 50 or system controller may comprise a processor 156 and a memory 158. The processor 156 may include one or more digital processing devices including, for example, a central processing unit (CPU) with one or more processing cores, a graphics processing unit (GPU), digital signal processors (DSPs), field programmable gate arrays (FPGAs), application specific integrated circuits (ASICs) and the like. In some configurations multiple processing devices are combined into a System on a Chip (SoC) configuration while in other configurations the processing devices may correspond to discrete components. In operation, the processor 156 may execute program instructions stored in the memory 158 to perform various operations related to the operation of the imaging system 150 as well as one or more surgical control consoles 160 in communication with the controller 50.

The memory 158 may comprise one or more data storage devices including, for example, magnetic or solid-state drives and random access memory (RAM) devices that store data. The memory 158 may include one or more stored program instructions, object detection templates, image processing algorithms, etc. In various implementations, the controller 50 may correspond to a display or video controller configured to output formatted image data to one or more display devices 162. In such applications, the controller 50 may include one or more formatting circuits 164, which may process the image data received from the camera apparatus 12 and/or the surgical scope 152, communicate with the processor 156, and process the image data for presentation on the one or more display devices 162. The formatting circuits 164 may include one or more signal processing circuits, analog-to-digital converters, digital-to-analog converters, etc. The user interface 154 of the controller 50 may be in the form of an integrated interface (e.g., a touchscreen, input buttons, an electronic display, etc.) or may be implemented by one or more connected input devices (e.g., a tablet) or peripheral devices (e.g., keyboard, mouse, foot pedal, etc.).

In various implementations, the system may comprise a surgical pump 166 (e.g., an infusion pump) configured to supply and/or control the flow of fluid to the sheath 55 via an inflow tube 168. In various implementations, the pump 166 may be in communication with the system 150 via one or more communication circuits (e.g., I/O circuits 172) of the control console 160 and/or the controller 50 of the camera apparatus 12 and/or the scope 152. The pump 166 may be configured to supply fluid to/from the surgical site to facilitate various procedures. In various implementations, the pump 166 may be controlled via the user interface 154 to adjust a flow rate, pressure, or intensity of the fluid transfer. The pump 166 may be implemented with a variety of pumping technologies (e.g., peristaltic, reciprocating, etc.) and may vary in fluid transfer capacity based on the application.

As shown, the controller 50 may also be in communication with an external device or server 170, which may correspond to a network, local or cloud-based server, device hub, central controller, or various devices that may be in communication with the controller 50 and, more generally, the imaging system 150 via one or more wired (e.g., serial, Universal Serial Bus (USB), Universal Asynchronous Receiver/Transmitter (UART), etc.) and/or wireless communication interfaces (e.g., a ZigBee, an Ultra-Wide Band (UWB), Radio Frequency Identification (RFID), infrared, Bluetooth®, Bluetooth® Low Energy (BLE), Near Field Communication (NFC), etc.) or similar communication standards or methods. For example, the controller 50 may receive updates to the various modules and routines as well as communicate sample image data from the camera apparatus 12 to a remote server for improved operation, diagnostics, and updates to the imaging system 150. The user interface 154, the external server 170, and/or a surgical control console 160 may be in communication with the controller 50 via one or more I/O circuits 172. The I/O circuits 172 may support various communication protocols including, but not limited to, Ethernet/IP, TCP/IP, Universal Serial Bus, Profibus, Profinet, Modbus, serial communications, etc.

According to some aspects of the disclosure, a surgical camera apparatus includes an elongated head assembly having a head profile extending along a longitudinal axis. The elongated head assembly forms an angled distal tip oriented at a tip angle offset from the longitudinal axis, and the head profile is elongated along a height of a distal face formed by a tip angle of the distal tip.

According to various aspects, the disclosure may implement one or more of the following features or configurations in various combinations:
wherein the distal tip is formed by an insert housing operably coupled within a tubular housing forming the elongated head, and wherein the distal tip is formed by a light-transmissive material through which light emitted from at least one light source illuminates a field of view of the camera apparatus;
the head profile forms an elongated cross-section that is greater in height than width;
the elongated cross-section of the head profile is further elongated along a distal face formed by the angled distal tip at the tip angle;
the tip angle is between is between 10° and 70° or between 15° and 45°;
the tip angle is approximately 25° to 35°;
the elongated head assembly is formed by a tubular housing forming an interior cavity, the tubular housing forms an outer surface of the elongated head assembly that extends along the head profile, and wherein an insert housing is retained within the tubular housing forming a sealed distal end about a perimeter of the distal tip of the housing;
a camera module is disposed within the insert housing and extends to a distal face of the distal tip;
a light source disposed within the interior cavity behind a portion of the insert housing relative to the distal face, wherein the light source emits a light through a light-transmissive portion for of the insert housing;
the camera module comprises an elongated housing comprising a distal lens and a proximal image sensor separated over a focal length, wherein the elongated housing is disposed within the interior cavity of the tubular housing;
a distal lens camera module and an emission surface of the light source are stacked within the cavity along an elongated dimension of the head profile;
the light source is positioned distally of the camera module on the distal face along the tip angle;
the proximal position of the camera module relative to the light source aligns a depth of the camera module diagonally through the cavity along the elongated dimension of the head profile;
a conductive connector extending through a length of the tubular housing interconnecting the camera module and the light source to a control console of the camera apparatus;
the tip angle is formed through the tubular housing along a distal face of the distal tip; and/or
the camera module and the light source are sealed within the tubular housing in a potted assembly.

According to another aspect of the disclosure, a surgical camera apparatus includes a camera module and a light source. An insert assembly forms a distal tip of the surgical camera apparatus to which the camera module and the light source are operably coupled. An elongated tubular housing receives the insert assembly, wherein the distal tip of the insert assembly comprises a perimeter ridge that engages a distal opening of the elongated tubular housing forming a sealed assembly between the tubular housing and the insert assembly.

According to various aspects, the disclosure may implement one or more of the following features or configurations in various combinations:
the insert assembly comprises an insert housing, and the camera module is secured within an aperture formed through the insert housing extending from the distal tip into an interior cavity formed within tubular housing;
the light source is coupled to an interior surface of the insert assembly and positioned within the sealed assembly, wherein an emission surface of the light source is directed to output a lighting emission through the distal tip of the insert assembly; and/or
the distal tip is formed by a light transmissive material through which the lighting emission is transmitted.

According to yet another aspect of the disclosure, a surgical camera apparatus includes an elongated head assembly having a head profile extending along a longitudinal axis. The elongated head assembly forms an angled distal tip oriented at a tip angle offset from the longitudinal axis and the head profile is elongated along a height of a distal face formed by a tip angle of the distal tip. The distal tip forms a distal face extending along a height of the head profile at the tip angle, and the height of the head profile is increased along the distal face as a result of the tip angle extending diagonally through the height of the head profile at the tip angle.

According to various aspects, the disclosure may implement one or more of the following features or configurations in various combinations:
the distal tip is formed by an insert housing operably coupled within a tubular housing forming the elongated head, and wherein the distal tip is formed by a light-transmissive material through which light emitted from at least one light source illuminates a field of view of the camera apparatus;
an elongated dimension of the distal face extending vertically along the height of the head profile forms a hypotenuse of the angled distal tip at the tip angle;
an elongated sheath forming a flow passage configured to receive the head profile;
the head profile forms an elongated cross-section that is greater in height than width and the height of the head profile extends over a cross-sectional flow path formed by the flow passage;
the cross-sectional flow path is circular and the height of the head profile substantially bisects the cross-sectional flow path separating the cross-sectional flow path to opposing sides of the head profile separated over the width;
the elongated head assembly is in connection with an interface connection that extends thorough a length of the elongated sheath, wherein the head profile occupies a first cross-sectional flow area within the flow passage and an interface profile of the connection interface occupies a second cross-sectional flow area within the flow passage, wherein the first cross-sectional flow area is greater than the second cross-sectional flow area; and/or
a proximal end portion of the elongated head assembly forms a transition region longitudinally drafted from the interface profile to the head profile.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A surgical camera apparatus comprising an elongated head assembly having a head profile extending along a longitudinal axis, wherein the elongated head assembly forms an angled distal tip oriented at a tip angle offset from the longitudinal axis and the head profile is elongated along a height of a distal face formed by a tip angle of the distal tip.

2. The surgical camera apparatus according to claim 1, wherein the distal tip is formed by an insert housing operably coupled within a tubular housing forming the elongated head, and wherein the distal tip is formed by a light-transmissive material through which light emitted from at least one light source illuminates a field of view of the camera apparatus.

3. The surgical camera apparatus according to claim 1 wherein the head profile forms an elongated cross-section that is greater in height than width.

4. The surgical camera apparatus according to claim 3, wherein the elongated cross-section of the head profile is further elongated along a distal face formed by the angled distal tip at the tip angle.

5. The surgical camera apparatus according to claim 1, wherein the tip angle is between is between 10° and 70° or between 15° and 45°.

6. The surgical camera apparatus according to claim 1, wherein the tip angle is approximately 25° to 35°.

7. The surgical camera apparatus according to claim 1, wherein the elongated head assembly is formed by a tubular housing forming an interior cavity, the tubular housing forms an outer surface of the elongated head assembly that extends along the head profile, and wherein an insert housing is retained within the tubular housing forming a sealed distal end about a perimeter of the distal tip of the housing.

8. The surgical camera apparatus according to claim 7, further comprising:
a camera module is disposed within the insert housing and extends to a distal face of the distal tip; and
a light source disposed within the interior cavity behind a portion of the insert housing relative to the distal face, wherein the light source emits a light through a light-transmissive portion for of the insert housing.

9. The surgical camera apparatus according to claim 8, wherein the camera module comprises an elongated housing comprising a distal lens and a proximal image sensor separated over a focal length, wherein the elongated housing is disposed within the interior cavity of the tubular housing.

10. The surgical camera apparatus according to claim 8, wherein a distal lens camera module and an emission surface of the light source are stacked within the cavity along an elongated dimension of the head profile.

11. The surgical camera apparatus according to claim 10, wherein the light source is positioned distally of the camera module on the distal face along the tip angle.

12. The surgical camera apparatus according to claim 11, wherein the proximal position of the camera module relative to the light source aligns a depth of the camera module diagonally through the cavity along the elongated dimension of the head profile.

13. The surgical camera apparatus according to claim 8, further comprising:
a conductive connector extending through a length of the tubular housing interconnecting the camera module and the light source to a control console of the camera apparatus.

14. The surgical camera apparatus according to claim 7, wherein the tip angle is formed through the tubular housing along a distal face of the distal tip.

15. The surgical camera apparatus according to claim 7, wherein the camera module and the light source are sealed within the tubular housing in a potted assembly.

16. A surgical camera apparatus comprising:
a camera module and a light source;
an insert assembly forming a distal tip of the surgical camera apparatus to which the camera module and the light source are operably coupled; and
an elongated tubular housing that receives the insert assembly, wherein the distal tip of the insert assembly comprises a perimeter ridge that engages a distal opening of the elongated tubular housing forming a sealed assembly between the tubular housing and the insert assembly.

17. The surgical camera apparatus according to claim 16, wherein the insert assembly comprises an insert housing, and the camera module is secured within an aperture formed through the insert housing extending from the distal tip into an interior cavity formed within tubular housing.

18. The surgical camera apparatus according to claim 16, wherein the light source is coupled to an interior surface of the insert assembly and positioned within the sealed assembly, wherein an emission surface of the light source is directed to output a lighting emission through the distal tip of the insert assembly.

19. The surgical camera apparatus according to claim 16, wherein the distal tip is formed by a light transmissive material through which the lighting emission is transmitted.

20. A surgical camera apparatus comprising an elongated head assembly having a head profile extending along a longitudinal axis, wherein the elongated head assembly forms an angled distal tip oriented at a tip angle offset from the longitudinal axis and the head profile is elongated along a height of a distal face formed by a tip angle of the distal tip, wherein the distal tip forms a distal face extending along a height of the head profile at the tip angle, and wherein the height of the head profile is increased along the distal face as a result of the tip angle extending diagonally through the height of the head profile at the tip angle.

21. The surgical camera apparatus according to claim 20, wherein the distal tip is formed by an insert housing operably coupled within a tubular housing forming the elongated head, and wherein the distal tip is formed by a light-transmissive material through which light emitted from at least one light source illuminates a field of view of the camera apparatus.

22. The surgical camera apparatus according to claim 20, wherein an elongated dimension of the distal face extending vertically along the height of the head profile forms a hypotenuse of the angled distal tip at the tip angle.

23. The surgical camera apparatus according to claim 20, further comprising an elongated sheath forming a flow passage configured to receive the head profile.

24. The surgical camera apparatus according to claim 23, wherein the head profile forms an elongated cross-section that is greater in height than width and the height of the head profile extends over a cross-sectional flow path formed by the flow passage.

25. The surgical camera apparatus according to claim 24, wherein the cross-sectional flow path is circular and the height of the head profile substantially bisects the cross-sectional flow path separating the cross-sectional flow path to opposing sides of the head profile separated over the width.

26. The surgical camera apparatus according to claim 23, wherein the elongated head assembly is in connection with an interface connection that extends thorough a length of the elongated sheath, wherein the head profile occupies a first cross-sectional flow area within the flow passage and an interface profile of the connection interface occupies a second cross-sectional flow area within the flow passage, wherein the first cross-sectional flow area is greater than the second cross-sectional flow area.

27. The surgical camera apparatus according to claim 26, wherein a proximal end portion of the elongated head assembly forms a transition region longitudinally drafted from the interface profile to the head profile.
